(19) United States
(12) Patent Application Publication
(10) Pub. No.: US 2003/0224994 A1
(43) Pub. Date: Dec. 4, 2003

(54) Storage stable liquid sealer protein complex

(76) Inventors: Colin John Newton, San Gabriel, CA (US); Thomas Seelich, Vienna (AU)

Correspondence Address: Baxter Healthcare Corporation, P.O. Box 15210, Irvine, CA, 92614, US

(21) Appl. No.: 10/407,699

(22) Filed: Apr. 4, 2003

Related U.S. Application Data
(60) Provisional application No. 60/370,600, filed on Apr. 5, 2002.

(51) Int. Cl. A61K 38/36; A61K 31/727

(52) U.S. Cl. 514/12; 514/56

(57) ABSTRACT

The present invention provides storage stable liquid sealer protein complexes containing fibrinogen that are convenient to store and transport, that are stable over several months, and that do not have to be discarded if an intended surgical procedure is not carried out. The complexes of the invention comprise fibrinogen, an anti-proteolytic agent, an anti-gelling agent, and a physiologically acceptable carrier.

### CROSS-REFERENCES TO RELATED APPLICATIONS

This application claims priority to U.S. provisional application Serial No. 60/370,600 filed Apr. 5, 2002.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Fibrinogen containing compositions have long been used as a physiological adhesive in therapeutic and surgical procedures. Such compositions are known as “sealer protein complexes.” Fibrinogen containing compositions are employed, for example, for assisting wound healing, for sealing large-area bleeding, for uniting tissue in parenchymal organs, for conjunctival bonding or for securing sutures, and for stopping bleeding after surgical interventions and other therapeutic procedures. Fibrinogen is also known to be useful for tissue adhesion, i.e., stable wound closure. Advantages of fibrinogen include its biocompatibility and naturally occurring resorption.

In tissue adhesion procedures with fibrin sealants, the final phase of blood coagulation is imitated. Specifically, sealer protein or fibrinogen complex, which contain factor XIII, is brought into contact with thrombin and calcium ions on the area to be bonded. Thrombin cleaves the fibrinopeptides A and B from fibrinogen, resulting in fibrin monomers which form a soluble fibrin clot. The transglutaminase factor XIII is simultaneously activated by thrombin and calcium ions, thus stabilizing the clot by crosslinking fibrin monomers. An increased resistance of the clot to fibrinolysis can be achieved by adding a plasmin inhibitor to the fibrinogen or thrombin solution.

Methods of preparing fibrinogen concentrates have been described (see, e.g., U.S. Pat. Nos. 4,650,678; US 4,362,567; US 4,298,598; US 4,377,572; EP 0 103 196, and EP 0 085 923). U.S. Pat. No. 4,650,678 describes a solid fibrinogen lyophilizate which is not cryoprecipitated. In another example, EP 0 103 196 describes the preparation of a fibrinogen concentrate from cryoprecipitate which, after thawing and dilution, has been treated with 2.5% Al(OH)₃. The concentrate is freeze-dried for stabilization and is stable for 4 hours after reconstitution. EP 0 085 923 describes the preparation of an arginine-containing fibrinogen concentrate which remains stable for a working day. It is necessary to dissolve the fibrinogen in as high a concentration as possible, since the adhesive action is a function of the fibrinogen concentration. Highly concentrated fibrinogen lyophilates require dissolution at elevated temperatures, typically 37° C., before their use. The use of deep-frozen fibrinogen cryoprecipitate is associated with disadvantages because such fibrinogen preparations are unstable and must be stored below −20° C. until use.

A fibrinogen lyophilizate has been disclosed in German Offenlegungsschrift No. 3,002,934. This lyophilizate is also a cryoprecipitate, which accordingly contains not only fibrinogen and factor XIII, but also plasminogen, albumin and other plasma constituents. An inhibitor of plasminogen activator is added to stabilize the lyophilizate and the reconstituted solution. This lyophilizate dissolves slowly at elevated temperature (37° C.). After reconstitution, the product is stable for a maximum of 4 hours at room temperature.

Commercial products are available for tissue adhesion with adhesives containing fibrin and are described in U.S. Pat. Nos. 6,277,961 B1; US 5,962,405; US 4,362,567; US 4,298,598; US 4,909,251; and US 4,377,572. The commercially available fibrin adhesives are two-component adhesives: (1) fibrinogen, factor XIII and aprotinin and (2) thrombin and calcium ions. The components are in either deep-frozen or freeze-dried form. One disadvantage of these products is the need to prepare the adhesive for each use, since both the thawing, warming, and dissolving of the lyophilizates may take 20-30 minutes and involve multiple steps. Complicated reconstitution could impact the sterility of the fibrinogen. Another considerable disadvantage is the relatively short stability (about 4 hours) of the adhesive components once they are ready for use. The material may be prepared ahead of time but if the fibrin sealant is not subsequently needed it can lose its activity and must be discarded, which increases expense. Moreover, reconstitution requires that additional staff be present, adding to the costs of procedures requiring fibrin adhesives.

The user may prepare fibrin adhesives as described in WO 86/01814. The user can also prepare fibrinogen concentrates according to methods known in the art, and combine the concentrates with commercially obtainable thrombin products to form a tissue adhesive. The procedure described in WO 86/01814 is, however, associated with a disadvantage: to prevent transmission of viruses, only blood from the patient to be treated can be used for obtaining the fibrinogen concentrate. Preparation of the concentrate is time-consuming, and thus, application is not possible in cases of emergency. Moreover, consistent composition and quality of the fibrinogen concentrate is not ensured when plasma from a single donor is used.

Because of the disadvantages of currently available fibrinogen preparations and user prepared fibrinogen concentrates, there is a need in the art for storage stable liquid fibrinogen solutions which have maximum stability in a form immediately ready for use. A storage stable liquid fibrinogen solution could eliminate preparation time required to reconstitute currently marketed lyophilized products, reduce costs, and increase convenience. The handling required to use such a product would be greatly reduced, thereby increasing the assurance that the product's sterility would be maintained while treating the patient.

### BRIEF SUMMARY OF THE INVENTION

This invention provides novel compositions of storage stable liquid fibrinogen solutions (i.e., protein sealer complex) and novel methods of making storage stable liquid protein sealer complex. The storage stable liquid protein sealer complex of the invention has multiple advantages over fibrinogen concentrates known in the art. For example, the storage stable liquid protein sealer complex of the invention is more convenient because no time needs to be spent thawing or dissolving the fibrinogen component. The storage stable liquid protein sealer complex of the present invention is also stable at 15-25° C. over a lengthy period of time and does not have to be discarded if an intended therapeutic procedure is not carried out. The liquid sealer protein is stable for about a month at 5° C., which enables it to be transported in cold conditions without gelling or undergoing proteolysis. One advantage of this composition is that it is ready for use without the need for a reconstitution step; this relieves surgical staff from the need to exert extra care in preventing the introduction of pathogens into the protein sealer complex.

One embodiment of the present invention provides a storage stable fibrinogen composition (protein sealer complex) in an aqueous solution. The fibrinogen composition comprises fibrinogen in the form of protein sealer complex; an anti-proteolytic agent; an anti-gelling agent; and an antioxidant agent.

Another embodiment of the present invention provides a method of making a storage stable liquid protein sealer complex.

### BRIEF DESCRIPTION OF THE DRAWINGS

Not applicable.

### DETAILED DESCRIPTION OF THE INVENTION

I. Introduction

The present invention provides storage stable liquid protein sealer complex that has multiple advantages over fibrinogen concentrates known in the art. For example, the storage stable liquid protein sealer complex of the invention is more convenient because no time needs to be spent thawing or dissolving the fibrinogen component. The storage stable liquid protein sealer complex of the present invention is also stable over a lengthy period of time and does not have to be discarded if an intended surgical procedure is not carried out. Storage stable liquid protein sealer complex of the present invention is stable at 15-25° C. for at least 6 months and preferably longer without losing its activity.

II. Definitions

“Fibrinogen” may be recombinant or naturally occurring. The term “fibrinogen” also refers to polypeptide polymorphic variants, alleles, mutants, and interspecies homologues of fibrinogen. The terms “fibrinogen,” “fibrinogen solution” and “protein sealer complex” are used interchangeably herein. In a preferred embodiment, a fibrinogen solution also comprises other components, such as, for example, Factor XIII, fibronectin, plasminogen, cold-insoluble globulin, plasminogen activator inhibitor, plasmin inhibitor, niacinamide, or human serum albumin. Means of preparing fibrinogen from citrated plasma are taught, for example, in U.S. Pat. Nos. 4,650,678; US 5,773,033; US 6,277,961; and US 4,362,567. A method of producing fibrinogen in transgenic mammals is disclosed in U.S. Pat. No. 5,639,940. A method of preparing fibrinogen from autologous plasma is disclosed in U.S. Pat. No. 5,773,033.

A “reduced level” of plasminogen refers to levels of plasminogen at least 70%, preferably 75, 80, 85, 90, or 95%, most preferably 98% below endogenous levels of plasminogen. “Endogenous” levels of plasminogen refers to levels of plasminogen found in a reconstituted protein sealer complex prepared from lyophilized fibrinogen which was extracted by precipitation from plasma.

An “anti-proteolytic agent” is any agent or compound or compounds that prevent or reduce fibrinogen proteolysis. The anti-proteolytic agent may act by inhibiting or neutralizing the protease.

An “anti-gelling agent” is any agent or compound or combination of compounds that prevent or reduce fibrinogen gelation. Gelation occurs when the sealer protein is stored at cooler temperatures, especially around 5° C., and subsequently, upon warming above its storage temperature, the protein solution does not liquefy, i.e., the liquid fibrinogen solution forms a solid precipitate or a gel.

An “antioxidant agent” is any agent or compound or compounds that prevent or reduce oxidation of a protein sealer complex, i.e., prevent or reduce oxygen induced chemical damage to proteins in a liquid state. For example, an antioxidant agent may be a reducing agent such as, for example, glutathione or cysteine. An antioxidant agent may also be a layer of an inert gas such as nitrogen overlaying the solution, thus preventing oxygen from entering the solution.

The terms “storage stable” and “storage stability” are used interchangeably to refer to the ability of a fibrinogen solution to resist proteolysis, maintain structural integrity, and maintain functionality (clotting ability and resistance to oxidation) and sterility under storage at temperatures from around 15-25° C. over a period of time. Storage stability can be enhanced by addition of protease inhibitors with or without removal of endogenous plasminogen. Reduction or removal of endogenous plasminogen also enhances storage stability. The storage stable liquid fibrinogen solution of the present invention has at least 6 months storage stability and preferably a longer duration of storage stability.

A “therapeutically effective amount” of a storage stable liquid sealer protein complex is an amount sufficient to provide a therapeutic effect, i.e., an amount of storage stable liquid sealer protein complex effective for sealing large-area bleeding, for uniting tissue in parenchymal organs, for conjunctival bonding or for securing sutures, or for stable wound closure and stopping bleeding after various surgical interventions and other medical procedures.

III. Fibrinogen Composition of the Invention

One embodiment of the present invention is a storage stable liquid sealer protein complex that includes fibrinogen complex, an anti-proteolytic agent, an anti-gelling agent, and an antioxidant agent. The liquid sealer protein complex may have reduced levels of plasminogen. The liquid sealer protein complex may be depleted of oxygen by any means known in the art such as, for example by degassing and purging with N₂, or by the addition of an antioxidant agent, or both.

Fibrinogen is present in the solution at a concentration of 30 mg/ml, preferably 50 mg/ml, more preferably 70 or 80 mg/ml, e.g., a range of about 30-130 mg/ml.

A. Preparation of Fibrinogen

Fibrinogen for the composition of the present invention can be prepared using any means known in the art. Means of preparing fibrinogen complex from citrated plasma are taught, for example, in U.S. Pat. Nos. 4,650,678, 5,773,033, and 6,277,961 B1. A method of producing fibrinogen in transgenic mammals is disclosed in U.S. Pat. No. 5,639,940. A method of preparing fibrinogen from autologous plasma is disclosed in U.S. Pat. No. 5,773,033. Methods for producing fibrinogen-containing preparations which can be used as tissue adhesives or fibrin sealants have been described. (See, e.g., U.S. Pat. Nos. 5,792,835; US 5,716,645; US 4,377,572; US 4,362,567; US 4,909,251; Methods of Plasma Protein Fractionation (1980) Curling ed. and Blomback, Arkiv Kemi (1959) 10:415, et seq.). EP 0103196 describes preparation of a fibrinogen concentrate from cryoprecipitate which, after thawing and dilution, has been treated with 2.5% Al(OH)₃. EP 0 085 923 describes preparation of an arginine-containing fibrinogen concentrate which remains stable for a working day. A fibrinogen lyophilizate which contains fibrinogen, factor XIII, plasminogen, albumin and other plasma constituents has been disclosed in German Offenlegungsschrift No. 3,002,934.

B. Anti-Proteolytic Agents

At least one anti-proteolytic agent is required as a component of the storage stable liquid sealer protein complex solutions of the present invention. One suitable anti-proteolytic agent is alpha 2 antiplasmin, which can be used as the only anti-proteolytic agent, or it can be used in combination with the plasmin inhibitor, aprotinin. Alternatively, in combination with aprotinin, the anti-proteolytic agent may be alpha 1 protease inhibitor, antithrombin III plus heparin, or combinations thereof. When alpha 2 antiplasmin is used as the only anti-proteolytic agent, its preferred concentration is about 5-10 μM. When alpha 2 antiplasmin at about 5-10 μM is used in combination with aprotinin, the preferred concentration of aprotinin is about 40-120 μM. Another preferred anti-proteolytic agent is alpha 1 protease inhibitor at a concentration of about 20-50 μM, used in combination with aprotinin at about 40-120 μM. Another preferred combination is antithrombin III at a concentration of about 20-50 μM plus heparin at about 10-25 μM, used in combination with aprotinin at about 40-120 μM. When using antithrombin III, it is preferred to use heparin at about half the concentration of antithrombin III because of the mechanism of action. Heparin binds to lysyl residues on antithrombin and dramatically accelerates its rate of binding to thrombin (Rosenberg, R. D. and Bauer, K. A., Chapter 41, pages 837-838, In: Hemostasis and Thrombosis: Basic Principles and Clinical Practice, third edition, edited by Robert W. Colman, et al; J. B. Lippincott Company, Philadelphia, 1994). Heparin is thought to do this in a catalytic fashion and therefore its concentration may be set lower than that of antithrombin III.

C. Anti-Gelling Agents

Anti-gelling agents are one component of the storage stable liquid sealer protein complex solutions of the present invention. Suitable anti-gelling agents include, for example arginine, histidine, and urea. Preferably the anti-gelling agent is arginine. The anti-gelling agent may be present in a concentration from about 1 mM to about 500 mM, preferably from about 5 mM to about 250 mM, more preferably from about 10 mM to about 100 mM, most preferably from about 40 mM to about 50 mM. In a preferred embodiment, the composition comprises 40-50 mM arginine neutralized to pH 7.3 with citric acid.

D. Antioxidant Agents

An antioxidant is one component of the storage stable liquid sealer protein complex solutions of the present invention. Suitable antioxidant agents include, for example, glutathione and cysteine. The antioxidant agent may be added to bring it to an initial concentration of 2-5 mM. Preferably the antioxidant agent is glutathione. The storage stable liquid fibrinogen solutions may also be stored under an inert gas such as nitrogen, argon or helium, to maintain a low oxygen content in the storage stable liquid sealer protein complex solution. The inert gas is preferably a non-noble inert gas, a noble gas, or more preferably nitrogen.

IV. Making the Storage Stable Liquid Sealer Protein Complex Solutions

The storage stable liquid sealer protein complex solutions of the present invention may be made by reconstituting sealer protein complex in an aqueous solution comprising an anti-proteolytic agent, an anti-gelling agent and an antioxidant agent. The solution may be degassed and treated with nitrogen gas after reconstitution. The sealer protein may be treated with UV light before degassing, to inactivate virus, in addition to solvent/detergent treatment and vapor heating steps for inactivating virus which are known in the art.

A. Reconstitution of Fibrinogen

Fibrinogen may be reconstituted by any means known in the art. Methods of reconstituting lyophilized or cryoprecipitated fibrinogen are described in U.S. Pat. Nos. 5,962,405 and 6,277,961 B1. A liquid fibrinogen solution may also be made by reconstituting purified fibrinogen with 20-50 mM arginine neutralized with citric acid (pH 7.3) for anti-gelling purposes according to the present invention.

The solubility of the fibrinogen may be improved or enhanced by the addition of solubility modifiers such as, for example, vitamins; aromatic compounds, such as, for example, compounds derived from benzene or phenol or those derived from heterocyclic compounds, such as, for example, piperidine, pyridine, or pyrimidine; alcohols; such as, for example, ethanol; salts, such as, for example, inorganic salt, organic salt, ammonium salts, or alkali salts; polyols such as, for example, polyalkylene glycol or polyether; amino acids such as, for example, glycine or β-alanine; ethers; ketones; organic polymers, such as, for example, polyethylene glycol. Preferably the liquid fibrinogen solution has about 25%, more preferably 30%, most preferably 40% higher solubility after addition of these components.

The viscosity of the fibrinogen may be improved or reduced by addition of viscosity modifiers such as, for example, substances containing urea or guanidine residues, niacinamide, or high salt concentrations. Preferably the liquid fibrinogen solution has about 25%, more preferably 30%, most preferably 40% lower viscosity after addition of these components. In a preferred embodiment, niacinamide is added to reduce viscosity of the sealer protein (see, e.g., U.S. Pat. No. 5,962,405).

Plasminogen may be reduced or removed from the reconstituted fibrinogen solution using techniques known in the art such as, for example, lysine-Sepharose chromatography or by binding the fibrinogen to DEAE-cellulose, lowering the pH of the anion exchanger to pH 6.5, and eluting the plasminogen with 0.2-0.5 M tranexamic acid.

Prothrombin may be reduced or removed from the reconstituted fibrinogen solution using calcium phosphate treatment of the sealer protein. Other serine proteases such as elastase may be removed in order to further enhance storage stability of the final formulation.

The presence of sufficient Factor XIII is necessary to preserve clot elasticity, as the function of Factor XIII is to cross-link the clot. At the time that the sealer protein is to be used, it preferably contains at least 3 U of Factor XIII per 100 mg of sealer protein. Since Factor XIII activity declines over time, supplemental Factor XIII may be added to the sealer protein at the time of formulation so that even with some loss of activity, sufficient Factor XIII remains at the time of use to support cross-linking of the clot. It is preferable that the sealer protein contain at least 10 U of Factor XIII at the time of formulation.

As described above, suitable anti-proteolytic agents, anti-gelling agents, and antioxidant agents, are added to the reconstituted fibrinogen solution, to produce storage stable liquid sealer protein complex.

B. Deoxygenation of the Liquid Sealer Protein Complex Solution

The fibrinogen-containing solutions of the present invention may be maintained oxygen-free by one or a combination of the following: (1) initially degassing the solutions under vacuum followed by purging with oxygen-free nitrogen, and (2) adding glutathione or cysteine at about 2-5 mM. Methods of degassing solutions are well known to those of skill in the art.

C. Sterilizing the Liquid Sealer Protein Complex Solution

Preparation of the storage stable liquid sealer protein complex solution may comprise a sterilization, i.e., pathogen inactivation step. Any method known in the art for pathogen inactivation may be used. Suitable methods include, for example dry heat and vapor heat treatment (see, EP 0 159 311, EP 0 519 901, or EP 0 674 531), chemical treatment, such as for example with solvent or detergent or a combination of the solvent and detergent (see, WO 94/13329, DE 4434538, or EP 0131 740); physical treatment, such as, for example, filtration; photo inactivation with, for example visible (see, e.g., U.S. Pat. No. 5,922,278) or ultraviolet light. Each of the aforementioned sterilization methods can be used alone or in combination with one or more of the other enumerated methods of sterilizing storage stable liquid fibrinogen solutions. If used in combination, the sterilization methods can be performed simultaneously or sequentially.

Photo inactivation is particularly preferred for enhancing the safety of the liquid sealer protein complex solution product by inactivating heat resistant non-lipid enveloped viruses, e.g., parvovirus, or hepatitis viruses, without denaturing the proteins. It is desirable that exposure of the storage stable liquid sealer protein complex solution to a light source provide maximum viral inactivation with minimal damage to the fibrinogen solution. Also, it is desirable that exposure of the storage stable liquid sealer protein complex solution to a light source be of short duration to increase efficiency of treatment and reduce the cost. It is further desirable that the exposure of the storage stable liquid sealer protein complex solution to the light source be substantially uniform in its application and preferably without the need to continuously mix the components. It is further desirable to be able to treat more than one unit or container of liquid sealer protein complex solution at a time to improve efficiency, but without adverse effect on the outcome of the viral inactivation.

D. Packaging

Packaging of the storage stable sealer protein is designed to prevent oxidation, to promote water retention, and to promote ease of application. As described above, oxygen removal is part of the formulation and finishing process. To further prevent oxidation, the inclusion in the package of oxygen absorbing chemicals such as Ageless® sachets (Mitsubishi Gas Chemical Company, Inc.) ensures that the product is maintained oxygen free. It is preferred that the liquid product be presented to the surgeon in prefilled syringes, thus the syringes and packaging must be capable of retaining the water in the formulation. A humidified storage pouch could alleviate this potential problem. For ease of application, viscous drag of the liquid against the walls of the syringe should be kept to a minimum. As described in U.S. Pat. No. 5,962,405, the addition of niacinamide to the formulation is known to reduce viscosity. A coating such as Parylene® on the inside of the syringe may further reduce viscous drag.

V. Administration of the Storage Stable Liquid Sealer Protein Complex Solution

A therapeutically effective amount of the storage stable liquid sealer protein complex solution of the present invention can administered by any means known in the art (see e.g. U.S. Pat. Nos. 4,359,049; US 4,631,055; US 4,735,616; and EP 292472). Suitable means of administration include, for example, topical application of the sealer protein complex solution by, for example, aerosol spray or by catheter. The sealer protein complex solution may also be incorporated, for example, into a gauze pad, a sponge, or a collagen gel-type matrix, such as, for example, a film, microbeads, or flakes, before topical application.

The storage stable liquid sealer protein complex solutions of the present invention may be administered alone or in conjunction with other components such as, for example, thrombin, fibronectin, Factor XIII, alpha 2 macroglobulin, alpha 1 antitrypsin, plasmin activator 1, plasmin activator 2, or a source of calcium ions. The additional components may be administered simultaneously or sequentially with the storage stable liquid sealer protein complex solutions.

The following examples are provided by way of illustration only and not by way of limitation. Those of skill will readily recognize a variety of non-critical parameters that could be changed or modified to yield essentially similar results.

### EXAMPLES

### Example 1

### Materials and Methods

Clotting Rate Assay: Clotting rate assays were used to study the coagulation rate of the liquid sealer protein complex solution samples, which had been kept deoxygenated over time as described below. After the addition of thrombin and calcium chloride, the turbidity of the sample at 350 nm was recorded using the Cary 300 UV/Vis spectrophotometer. Stability samples were diluted in 0.05 M Tris, pH 7.4, to a final concentration of 0.25 mg/mL. A sample volume of 1 mL was added to a disposable cuvette. The samples were pre-incubated at 37° C. for 4 minutes. 60 μL of 20 mM CaCl₂ was added to each sample. After 1 minute, 60 μL of 1 U/mL thrombin was added and the absorbance recorded for 30 minutes at 37° C.

Clot Appearance Assay: Clot appearance assays were used to observe the appearance of the clot during formation and the speed at which the clot could obscure an “X” printed on paper when observed through the top to the bottom of the vial. The solutions were pre-warmed to 37° C. using the Fibrinotherm. 0.3 mL of the liquid sealer protein complex solutions (50 mg/mL) and 0.3 mL of thrombin (4 U/mL) was pipetted into a vial. The vial was immediately vortexed and put back into the Fibrinotherm. After one minute the vial was placed over a letter X printed in bold, Ariel 16 pt font. The appearance was scored, the vial put back into the Fibrinotherm, and reassessed every 30 seconds. Scoring: 0 indicates that the clot was transparent from above and laterally, clear to slightly opaque. 1 indicates that the clot was transparent from above, and laterally but significantly opaque. 2 indicates that the clot was transparent from above but significantly opaque and the X was blurred. 3 indicates that the clot was opaque and the X was not visible. Stage 3 should typically be reached in three minutes. The clot should also appear white, otherwise this is an indication of altered clot structure.

Stability Studies: Stability studies typically used 50-80 mg/mL liquid sealer protein complex solutions containing 50 μg/mL gentamycin sulfate. Gentamycin sulfate was used to maintain sterility of the solution for feasibility studies, but would not be used in the manufactured product. Sample preparation was performed in a sterile hood, using either sterile 1 mL syringes or Eppendorf tubes.

Plasminogen Activation Assay: Samples were diluted to 0.1 mg/mL fibrinogen with deionized water. Streptokinase was added, at a final concentration of 40 U/mL, to the samples. The samples were incubated at 37° C. for 15 minutes after which time 100 μL of each sample were added to the wells of a microtiter plate. 50 μL of S-2403 substrate were added to each well and the rates of the reaction were recorded at 405 nm for 1 hour.

Clot elasticity assay: The elasticity of the clots was measured in a thromboelastograph analyzer (Haemoscope) according to the device manual.

Oxygen removal: Liquid sealer protein samples, in microcentrifuge tubes, were degassed under vacuum for 15 minutes and then purged with nitrogen (Praxair Ultra high purity grade 5.0, minimum purity 99.998%) for 1 hour. The samples were closed and transferred to airtight containers for storage with an Oxygen Absorber (Mitsubishi Gas Chemical Company, Inc., Ageless Oxygen Absorber, ZPT-100EC) and an Oxygen Indicator (Mitsubishi Gas Chemical Company, Inc., Ageless-Eye CS-7). The containers were degassed for 15 minutes and purged with nitrogen for 30 minutes. The indicator turns blue in greater than 0.5% oxygen. Oxygen in the container headspace was measured using the Foxy Fiber Optic Oxygen Sensor.

### Example 2

### Clot Dissolution Study

The effects of varying concentrations of alpha 2 antiplasmin (A2AP) on the stability of clots was determined. Fibrinogen was reconstituted to a final concentration of 100 mg/mL with 40 mM Citrate/Arginine buffer pH 7.0. Thrombin was reconstituted in 40 mM CaCl₂ with concentrations of A2AP ranging from 0.2 μM to 3.75 μM. Plasma, used as the incubation medium, was prepared by adding 50 μg/mL gentamycin sulfate, to maintain sterility, and 100 U/mL (50,000 U total) streptokinase, to activate plasminogen.

Fibrinogen clots were prepared in 2 mL syringes by adding 500 μL of 100 mg/mL fibrinogen to 500 μL of 500 U/mL thrombin containing A2AP. Syringes were connected to one another by tubing in order of increasing concentration of A2AP and then attached to a peristaltic pump. 500 mL of the plasma incubation medium was recycled through the syringes at a rate of 2 mL per minute. Photographs of the clots were taken daily until the clots were degraded. On day three, 24,000 units of streptokinase were added, and on days 4 and 8, 10,000 units of Urokinase were added to the plasma to activate plasminogen.

Clots formed in the presence of A2AP were extremely resistant to plasmin-induced lysis. The storage stable liquid sealer protein complex solution of the present invention was also stabilized by A2AP.

After one month the proteins in samples containing less than 1 μM A2AP were degraded. The proteins in samples containing higher concentrations of A2AP were relatively intact after 6 months.

Clots were made with the same concentrations of A2AP (0-3.75 μM) to test if the clots would lyze eventually. The clots were perfused with human plasma in the presence of streptokinase and urokinase. Generally samples containing lower concentrations of A2AP dissolved faster while clots containing higher concentrations of A2AP lasted longer. However, the clots did lyze eventually.

### Example 3

### Identification of Useful Anti-gelling Agents

A number of different compounds were added to the fibrinogen solution to determine whether they were useful for preventing gelation when the fibrinogen solution was incubated in the refrigerator (2-8° C.).

Arginine was found to be useful as an anti-gelling agent for the fibrinogen solution. Higher concentrations of histidine, lower concentrations of citrate with 0.1 M arginine, or urea were also found to be useful anti-gelling agents. Arginine at 50 mM was found to be useful for stabilizing the fibrinogen solution against cold gelation. Urea at 0.2 M was found to be useful for stabilizing the fibrinogen solution against cold gelation. From about 0.05 M to about 0.1 M arginine, combined with low concentrations of citrate, was found to be useful as an anti-gelling agent. Thus, depending on storage conditions, 20-50 mM arginine, pH 7.3 neutralized with citric acid, is useful for compositions of the present invention.

### Example 4

### Resistance to Proteolysis

The ability of 5 μM A2AP to prevent or reduce fibrinogen proteolysis after degassing and purging with nitrogen was tested. After four months the nitrogen-purged sample with glutathione retained the same clotting rate as the frozen control sample.

The ability of 5 μM A2AP, C1 esterase inhibitor (C1EI), alpha 1 protease inhibitor (A1PI), antithrombin III (ATIII) and antithrombin III combined with heparin to prevent or reduce fibrinogen proteolysis was tested. After four months of storage, 5 μM A2AP alone was shown to prevent or reduce fibrinogen proteolysis. Likewise, after four months of storage, ATIII plus heparin was shown to reduce or prevent fibrinogen proteolysis. C1E1 after three months of storage had reduced or prevented fibrinogen proteolysis.

### Example 5

### Effects of Anti-Proteolytics Under Long-Term Storage

Long-term stability studies were set up with samples containing approximately 80 mg/ml sealer protein with no albumin (Tisseele VH/SD bulk powder), 50 μg/ml gentamicin sulfate, 5 mM glutathione, degassed and nitrogen purged, and containing various inhibitors at various concentrations. The frozen control samples without added inhibitors showed no proteolysis over 9 months, as expected. The samples without inhibitors stored at both 25° C. and 30° C. showed intermediate to extensive proteolysis at 6 and 9 months as evidenced by loss of the Aα band of fibrinogen in SDS-PAGE gels. Inhibitors added:

1) Alpha 2 antiplasmin (ASAP) at 5 μM plus 5 mM glutathione: The samples remained as clear viscous liquids for 9 months at 25° C. At 6 months, the samples showed little proteolysis and good retention of the Aα band of fibrinogen. The protein concentrations showed no drop from target concentrations.

2) Alpha 1 protease inhibitor (A1PI) at 7, 14, and 21 μM plus aprotinin at 0.3 mg/mL (46 μM): The samples with inhibitors at both 25° C. and 30° C. remained clear viscous liquids for 9 months, except at the lowest A1PI concentration. SDS-PAGE showed little proteolysis by 6 months at 25° C. and the highest concentration of A1PI. There was no significant variation in protein concentration or pH over nine months. Clotting rates remained at 100% of control at 25° C. for 9 months. Clot elasticity remained stable at 25° C. and the highest A1P1 concentration.

3) C1 elastase inhibitor (C1EI) at 7, 14, and 21 μM plus aprotinin at 0.3 mg/mL: By 6 months at 25° C., there was loss of the Aα band of fibrinogen, even at the highest concentration of C1EI.

4) Antithrombin III (ATIII) at 7, 14, and 21 μM, plus heparin at 50% of the concentration of ATIII, plus aprotinin at 0.3 mg/mL: The samples with inhibitors remained as clear viscous liquids over the 9 months of the study. Analysis of SDS-PAGE runs indicated some loss of the Aα band of fibrinogen by 6 months. Clotting rate remained essentially 100% for 6 months at 25° C., once the thrombin added for the clotting assay was adjusted from 1 U/ml to 10 U/ml to allow for its inhibition by these particular inhibitors.

### Example 6

### Reducing Agents

Samples of sealer protein preserved by degassing and nitrogen purging plus either 2 mM glutathione, 2 mM ascorbic acid, or no further addition showed that glutathione preserved the clotting rate over 3 months at 25° C. but ascorbic acid did not. Subsequent studies showed that the optimum level of glutathione was about 5 mM.

### Example 7

### Preservation of Clot Elasticity

It was observed that samples stored over time lost clot elasticity, which could be restored by the addition of 3 U of Factor XIII per 100 mg sealer protein, which is the approximate amount of Factor XIII in the sealer protein at the beginning of the storage period. Thus, it is desirable to include supplemental Factor XIII in the final formulation of sealer protein to ensure that sufficient Factor XIII activity remains after storage, at the time the sealer protein kit is to be used.

It is understood that the examples and embodiments described herein are for illustrative purposes only and that various modifications or changes in light thereof will be suggested to persons skilled in the art and are to be included within the spirit and purview of this application and are considered within the scope of the appended claims. All publications, patents, and patent applications cited herein are hereby incorporated by reference in their entirety for all purposes.

What is claimed is:

1. A composition of a storage stable liquid sealer protein, the composition comprising: fibrinogen; an anti-proteolytic agent; an anti-gelling agent; an antioxidant agent; and a physiologically acceptable carrier.

2. The composition of claim 1, wherein the sealer protein has a reduced level of endogenous plasminogen.

3. The composition of claim 1, comprising about 60-110 mg/ml fibrinogen.

4. The composition of claim 1, wherein the anti-proteolytic agent is alpha 2 antiplasmin.

5. The composition of claim 4, wherein the alpha 2 antiplasmin is present at a concentration of about 5-10 μM.

6. The composition of claim 1, wherein the anti-proteolytic agent is a member selected from the group consisting of alpha 2 antiplasmin, antithrombin III plus heparin, and alpha 1 protease inhibitor, wherein the composition further comprises aprotinin.

7. The composition of claim 6, wherein the anti-proteolytic agent is alpha 1 protease inhibitor at a concentration of about 20-50 μM and aprotinin is present at a concentration of about 40-120 μM.

8. The composition of claim 6, wherein the anti-proteolytic agent is alpha 2 antiplasmin at a concentration of about 5-10 μM and aprotinin is present at a concentration of about 40-120 μM.

9. The composition of claim 6, wherein the anti-proteolytic agent is antithrombin III at a concentration of about 20-50 μM plus heparin at a concentration of about 10-25 μM, and aprotinin is present at a concentration of about 40-120 μM.

10. The composition of claim 1, wherein the anti-gelling agent is a member selected from the group consisting of: arginine, histidine, and urea.

11. The composition of claim 1, wherein the anti-gelling agent is arginine.

12. The composition of claim 11, wherein the arginine is present in a concentration of about 20 mM to about 50 mM, neutralized to pH 7.3 with citric acid.

13. The composition of claim 11, wherein the arginine is present in a concentration of about 45 mM to about 50 mM, neutralized to pH 7.3 with citric acid.

14. The composition of claim 1, wherein the antioxidant agent is glutathione or cysteine.

15. The composition of claim 1, wherein the antioxidant agent is glutathione.

16. The composition of claim 15, wherein the glutathione is present at a concentration of about 5 mM.

17. The composition of claim 1, wherein the composition has been depleted of oxygen by degassing and nitrogen purging.

18. A composition of a storage stable liquid sealer protein complex, the composition comprising: fibrinogen at a concentration of about 60-110 mg/ml; about 45-50 mM arginine, neutralized to pH 7.3 with citric acid; about 5 mM glutathione; about 5-10 μM alpha 2 antiplasmin; at least 3 U of Factor XIII per 100 mg of sealer protein; and a physiologically acceptable carrier.

19. A composition of a storage stable liquid sealer protein complex, the composition comprising: fibrinogen at a concentration of about 60-110 mg/ml; about 45-50 mM arginine, neutralized to pH 7.3 with citric acid; about 5 mM glutathione; about 5-10 μM alpha 2 antiplasmin; about 45-70 μM aprotinin; at least 3 U of Factor XIII per 100 mg of sealer protein; and a physiologically acceptable carrier.

20. A composition of a storage stable liquid sealer protein complex, the composition comprising: fibrinogen at a concentration of about 60-110 mg/ml; about 45-50 mM arginine, neutralized to pH 7.3 with citric acid; about 5 mM glutathione; about 20-30 μM alpha 1 protease inhibitor; about 45-70 μM aprotinin; at least 3 U of Factor XIII per 100 mg of sealer protein; and a physiologically acceptable carrier.

21. A composition of a storage stable liquid sealer protein complex, the composition comprising: fibrinogen at a concentration of about 60-110 mg/ml; about 45-50 mM arginine, neutralized to pH 7.3 with citric acid; about 5 mM glutathione; about 20-30 μM antithrombin III; about 10-15 μM heparin; about 45-70 μM aprotinin; at least 3 U of Factor XIII per 100 mg of sealer protein; and a physiologically acceptable carrier.

22. A method of making a storage stable liquid fibrinogen composition, said method comprising the step of: reconstituting protein comprising fibrinogen in an aqueous solution comprising an anti-proteolytic agent, an anti-gelling agent, an antioxidant, and a physiologically acceptable carrier.

23. The method of claim 22, further comprising the step of degassing the solution and N₂ purging.

24. The method of claim 22, further comprising the step of inactivating virus in the solution by treating said composition with UV light.

25. The method of claim 22, further comprising the step of adding Factor XIII to the composition.
